(19) United States
(12) Plant Patent Application Publication
(10) Pub. No.: US 2025/0344621 P1
(43) Pub. Date: Nov. 6, 2025

(54) IBERIS PLANT NAMED 'IBSZ0008'

(50) ;

(71) Applicant: Theodorus Cornelis Maria Van Kleinwee, Enkhuizen (NL)

(72) Inventor: Theodorus Cornelis Maria Van Kleinwee, Enkhuizen (NL)

(73) Assignee: SYNGENTA CROP PROTECTION AG, Basel (CH)

(21) Appl. No.: 19/238,670

(22) Filed: Jun. 16, 2025

(30) Foreign Application Priority Data
May 3, 2024 (QZ) 2024/1031

(51) Int. Cl. A01H 6/20; A01H 5/02

(52) U.S. Cl. CPC A01H 6/20; A01H 5/02
USPC PLT/263.1

(57) ABSTRACT

A new garden type Iberis plant named ‘IBSZ0008’ particularly distinguished by its medium to low vigor, mounded to upright plant habit, a medium size flower with white flower color, and is flowering in April to May in moderate climate conditions.

Latin name of the genus and species of the plant claimed: Iberis sempervirens.

Varietal denomination: ‘IBSZ0008’.

### BACKGROUND OF THE NEW PLANT

The present invention comprises a new Iberis, botanically known as Iberis sempervirens and hereinafter referred to by the variety name ‘IBSZ0008’.

‘IBSZ0008’ is a product of a planned breeding program. The new cultivar is medium to low vigor, has a mounded to upright plant habit, a medium size flower with white flower color, and is flowering in April to May in moderate climate conditions

‘IBSZ0008’ originated from a open pollination made in May 2018 in Enkhuizen, The Netherlands. Seeds were sown in September 2018 in Enkhuizen, The Netherlands and ‘ISZ0008’ was selected from the resulting population in May 2019 in Enkhuizen, The Netherlands.

The female parent was an unnamed proprietary seedling ‘14-M332’.

TABLE 1
Characteristics of the female parent, compared to ‘IBSZ0008’:

| | ‘IBSZ0008’ | ‘14-M332’ |
|---|---|---|
| Earliness: | April to May | 7-10 days earlier |
| Vigor: | Medium | Rather vigorous |
| Foliage color (upper): | RHS 147B | RHS 147A |

As ‘IBSZ0008’ results from an open pollination, the male parent is unknown.

The first act of asexual reproduction of ‘IBSZ0008’ was accomplished when vegetative stem cuttings were propagated from the initial selection in May 2019 in Enkhuizen, The Netherlands.

### BRIEF SUMMARY OF INVENTION

Horticultural examination of plants grown from cuttings of the plant initiated in May 2019 and continuing thereafter, has demonstrated that the combination of characteristics as herein disclosed for ‘IBSZ0008’ are firmly fixed and are retained through successive generations of asexual reproduction.

‘IBSZ0008’ has not been observed under all possible environmental conditions. The phenotype may vary significantly with variations in environment such as temperature, light intensity and day length.

A Plant Breeder's Right for this cultivar was applied for in the European Union on May 3, 2024, No. 2024/1031. ‘IBSZ0008’ has not been made publicly available more than one year prior to the filing of this application.

The following traits have been repeatedly observed and are determined to be basic characteristics of the new variety. The combination of these characteristics distinguishes this Iberis as a new and distinct variety.

### BRIEF DESCRIPTION OF THE PHOTOGRAPHS

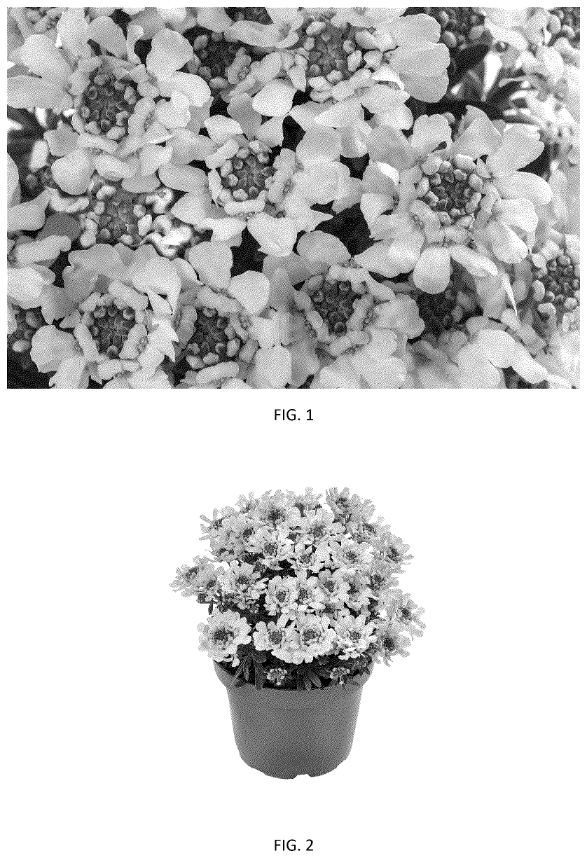

The accompanying photographic drawings show typical flower and foliage characteristics of ‘IBSZ0008’ with colors being as true as possible with an illustration of this type. The photographic drawing shows in FIG. 1 a close view of flower of the new variety and in FIG. 2 a flowering plant from an indoor trial.

The aforementioned photographs: FIG. 1, as well as FIG. 2, were taken on Mar. 24, 2023 both showing a plant from the same indoor trial in Enkhuizen, The Netherlands.

These plants were about 22 weeks of age. One rooted cutting per pot had been planted in a 12 cm pot, not pinched.

The measurements were taken in Enkhuizen, The Netherlands, in May 2025 on plants from an indoor trial.

### DETAILED BOTANICAL DESCRIPTION Color references are made to The Royal Horticultural Society Colour Chart (R.H.S.) 2001.

TABLE 2
DIFFERENCES BETWEEN THE NEW VARIETY
‘IBSZ0008’ AND A MOST SIMILAR VARIETY:

| | | ‘Snow Cone’, |
|---|---|---|
| | | U.S. Pat. No. |
| | ‘IBSZ0008’ | PP30,519 |
| Flowering time: | April-May | 10 days earlier |
| Foliage color: | RHS 147B | RHS 137A |
| Stem color: | RHS 144B | RHS 146B |

Plant:
   Form, growth and habit.—Hardy Perennial (can endure −20 C.). Dense, medium to low vigor, mounded to upright habit, very good branching.
   Plant height (above soil).—16 cm.
   Plant height (inflorescence included).—18 cm.
   Plant width.—20 cm.
Roots:
   Number of days to initiate roots.—About 9 days at about 22° C.
   Number of days to produce a rooted cutting.—18-23 days at 20° C.
   Type.—Fine, fibrous, moderate branching.
   Color.—RHS N155B.
Foliage:
   Arrangement.—Alternate.
   Immature leaf, color upper surface.—RHS 147B.
   Immature leaf, color lower surface.—RHS 147B.
   Mature leaf, color, upper surface.—RHS 147B.
   Mature leaf, color lower surface.—RHS 147B.
   Length.—3.0 on initial leaves, to 1.5 cm on leaves that develop later.
   Width.—0.2-0.5 cm.
   Shape.—Linear to slightly oblanceolate.
   Base shape.—Attenuate.
   Apex shape.—Rounded.
   Margin.—Entire.
   Texture, upper surface.—Glabrous.
   Texture, lower surface.—Glabrous.
   Color of veins, upper surface.—RHS 144B.
   Color of veins, lower surface.—RHS 144B.
   Pattern of veining.—Palmate.
   Petiole color.—No petiole.
Stem:
   Quantity of main branches per plant.—5.
   Color of stem.—RHS 144A, anthocyanin at base of stem, RHS N77A.
   Length of stem.—12 cm.
   Diameter.—0.4 cm.
   Length of internodes.—0.4-1.1 cm.
   Texture.—Rough surface, but no hairs.
   Color of peduncle.—RHS 146B.
   Length of peduncle.—0.5 cm, up to 4 cm when mature.
   Peduncle diameter.—0.3 cm.
   Texture.—Rough surface, but no hairs.
   Pedicel color.—RHS 146B.
   Pedicel length.—0.3-1.1 cm.
Inflorescence:
   Type.—Short simple corymb.
   Quantity of inflorescences per plant.—About 40.
   Lastingness of individual blooms on the plant.—10-14 days.
   Fragrance.—None.
BUD: (when showing color).
   Color.—RHS 1D.
   Length.—0.4 cm.
   Width.—0.5 cm.
   Shape.—Globose.
Immature inflorescence: (at moment of opening).
   Diameter.—3.3 cm.
   Color of ray florets, upper surface.—RHS 155D.
   Color of ray florets, lower surface.—RHS 155D.
Mature inflorescence:
   Diameter.—4 cm.
   Depth of floret.—1.0 cm.
   Diameter of floret.—1.3 cm.
   Shape of corolla.—4 petals; 2 small and 2 large, smaller and bigger petals are opposite of each other.
Ray florets:
   Average quantity of florets.—110.
   Color of florets, upper surface.—RHS 155D.
   Color lower surface.—RHS 155D.
   Length.—1.3 cm for the long petals, 0.8 for the short petals.
   Width/diameter.—0.5 cm for the long petals, 0.3 cm for the short petals.
   Shape.—Obovate.
   Apex shape.—Rounded.
   Margin.—Entire.
   Texture, upper surface.—Papillate.
   Lower surface.—Papillate.
Calyx:
   Type.—4 ovate shaped sepals with rounded apex, smooth surface and entire margin, fused at base.
   Length.—2-3 mm.
   Width.—2-3 mm.
   Colour (both upper and lower).—RHS 147C.
Reproductive organs:
   Pistil.—One.
   Length.—0.6 cm.
   Style color.—RHS 149D.
   Style length.—0.5 cm.
   Stigma color.—RHS 154C.
   Stigma shape.—Bi-parted.
   Ovary color.—RHS 149D.
   Ovary length.—0.1 cm.
   Ovary width.—0.1 cm.
Androecium:
   Stamens.—6.
   Color of filaments.—Base RHS 155C, RHS 148A near anther.
   Length filaments.—0.2-0.3 cm.
   Anther color.—RHS 5C.
   Anther length.—0.1 cm.
   Anther shape.—Oval to club shaped.
   Color of pollen.—RHS 4D.
   Pollen amount.—Moderate.
   Fertility/seed set.—Has not been determined to date.
   Disease/pest resistance.—Has not been determined to date.
   Hardiness.—Has not been determined to date.

What is claimed is:

1. A new and distinct variety of Iberis plant named ‘IBSZ0008’ substantially as illustrated and described herein.
